(19) United States
(12) Patent Application Publication
(10) Pub. No.: US 2015/0008127 A1
(43) Pub. Date: Jan. 8, 2015

(54) MEASURING APPARATUS AND METHOD

(71) Applicant: National Cheng Kung University, Tainan City (TW)

(72) Inventors: Shu-Hsien Liao, Taichung City (TW); Hsien Chang Chang, Tainan City (TW)

(21) Appl. No.: 14/323,824

(22) Filed: Jul. 3, 2014

(30) Foreign Application Priority Data
Jul. 4, 2013 (TW) 102124077

(51) Int. Cl. G01N 27/447; G01N 33/487; G01N 33/543

(52) U.S. Cl. CPC G01N 27/447; G01N 33/5438; G01N 33/48735; G01N 27/44756
USPC 204/547; 204/643

(57) ABSTRACT

An apparatus and method for measurement are disclosed. The apparatus and method separate solutes and suspended solids in a mixture using a dielectrophoretic force provided by an electrode and a conductive layer, and then perform quantitative or qualitative analysis on at least one of the solutes using the electrode or at least one of the suspended solids.

### CROSS-REFERENCE TO RELATED APPLICATION AND CLAIM OF PRIORITY

The application claims the benefit of Taiwan Patent Application No. 102124077, filed on Jul. 4, 2013, at the Taiwan Intellectual Property Office, the disclosures of which are incorporated herein in their entirety by reference.

### TECHNICAL FIELD

The present disclosure is directed to an apparatus and a method for measurement. The apparatus and method measure a specific property of a dissolved substance and/or a suspended substance in a sample which is pretreated using dielectrophoretic force.

### BACKGROUND

A dielectrophoresis chip uses the differences between dielectric properties among various kinds of particles in a liquid and creates a gradient in an AC field existing in the liquid to polarize the particles using a dielectrophoretic (DEP) force. Those particles having various dielectric properties will be driven by the positive DEP force to be attracted to the region with the strongest electric field intensity and driven by the negative DEP force to be excluded from the region with the weakest electric field intensity. Those particles are accordingly separated by the various dielectric properties.

Taiwan patent application No. 095131439 discloses a method to detect bioparticles in a biological sample (e.g. feces, urine, or other body fluid). Bioparticles (e.g. virus, bacteria and cell) often serve as carriers and indicators of pathogens and/or toxins. This method employs a substrate with interlaced comb-like electrodes on which a certain amount of sample mixed with antibodies-coated gold nanoparticles is dropped in the reservoir. Then the alternative signals with specific frequency bands are applied to the comb-like electrodes so that the Au-modified bioparticles, through DEP force, can be separated from the other constituents of the sample and can be attracted effectively to the edges of the electrodes. After washing the electrode surface with water to remove the residual sample several times, the device is measured for the impedance of the absorbed bioparticles on the edges of the electrodes. The measured impedance deviation in comparison with that of the reference empty comb-like electrodes will quantify the amount of the absorbed bioparticles.

After extensive experiments and persistent research, the applicant has finally conceived this measuring apparatus and the method thereof.

### SUMMARY

The present disclosure is directed to an apparatus and a method for measurement. The apparatus and method measure a specific property of a dissolved substance and/or a suspended substance in a sample which is treated with dielectrophoretic force.

In another aspect, the present disclosure discloses a method for measuring a concentration of a specific substance in a mixture including a plurality of particles, comprising steps of providing a single electrode having an initial indicator thereon; causing the mixture to be distributed on the single electrode so that the specific substance reacts with the indicator to cause the indicator to become a changed indicator; providing a dielectrophoretic force acting on the plurality of particles by the single electrode to drive at least a part of the plurality of particles away from a space directly above the single electrode; and obtaining the concentration of the specific substance via the changed indicator.

In another aspect, the present disclosure discloses a measuring apparatus, comprising a carrying portion carrying a liquid mixture, where the liquid mixture includes a non-dissoluble substance and a dissolved substance, the carrying portion includes an electrode having a surface directly contacting the liquid mixture and having an indicator thereon, and the indicator reacts with the dissolved substance; and a conductive layer directly contacting the liquid mixture, where the electrode and the conductive layer generate an AC field in the liquid mixture so as to cause the non-dissoluble substance to be distributed on the conductive layer, the dissolved substance reacts with the indicator to make the indicator become a changed indicator, and the changed indicator reflects a property of the dissolved substance.

In another aspect, the present disclosure discloses a measuring method, comprising steps of providing an electrode and a conductive layer, wherein at least one of the electrode and the conductive layer has a first indicator distributed thereon; providing a mixture including a suspended substance; distributing the mixture on the electrode and the conductive layer, wherein the suspended substance reacts with the first indicator to cause the first indicator to become a first changed indicator; generating an AC field in the mixture via the electrode and the conductive layer to drive the suspended substance away from a space directly above the electrode; and obtaining a property of the suspended substance based on the first changed indicator.

### BRIEF DESCRIPTION OF DRAWINGS

FIG. 1 is a diagram showing an embodiment of the present measurement system.

FIGS. 2a, 2b, 3a, 3b, 4a and 4b are diagrams showing embodiments demonstrating the separation of the suspended substance and the liquid portion of a liquid mixture.

### DETAILED DESCRIPTION

The present disclosure can be fully understood and accomplished by the skilled person according to the following embodiments. However, the practice of the present method is not limited to the following embodiments.

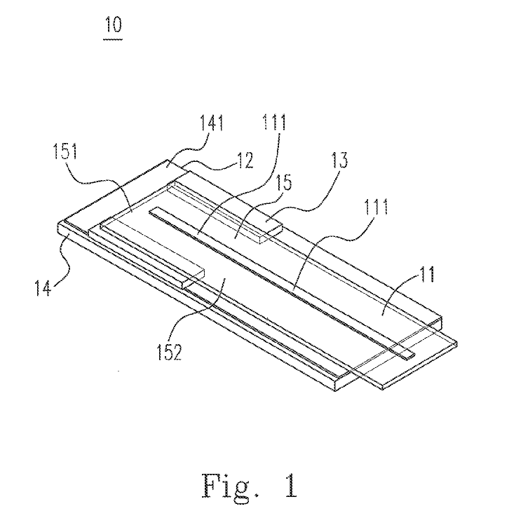

Please refer to FIG. 1 which shows an embodiment of the present measurement system. In FIG. 1, the measurement system is represented by a chip 10. Apparatus (chip) 10 includes a cover layer 11 and a substrate 12. Cover layer 11 has single electrode 111 disposed thereon, substrate 12 has a middle layer 13 and substrate layer 14, and a conductive layer 141 is configured on substrate layer 14.

In FIG. 1, a space 15 is surrounded by cover layer 11, middle layer 13, and substrate layer 14. Single electrode 111 and conductive layer 141 are located at the sides of space 15. Space 15 has a first opening 151 and a second opening 152 and is capable of containing a liquid mixture to be measured. When the liquid mixture contacts single electrode 111 and conductive layer 141 simultaneously, the liquid mixture, single electrode 111, and conductive layer 141 form a closed circuit so that an appropriate voltage can be applied to single electrode 111 and conductive layer 141 to obtain an electrical property of the liquid mixture. The electrical property can be further analyzed to reflect and determine other properties of the liquid mixture.

In some embodiments, single electrode 111 and conductive layer 141 are respectively located at two opposite sides of space 15. In addition, conductive layer 141 of chip 10 can be disposed on the entire surface of substrate 12, an entire side of space 15, or a partial side of space 15 to form the closed circuit with single electrode 111 and the liquid mixture.

When chip 10 is used to measure the properties of a dissolved substance and/or a suspended substance in the mixture, the mixture is distributed in space 15 first and directly contacts single electrode 111 and conductive layer 141. Next, when providing AC voltage to single electrode 111 and conductive layer 141, an AC field will be generated in space 15. Therefore, the suspended substance in the mixture contained in space 15 will be polarized by the AC field. The polarized suspended substance will be rapidly driven to move to a specific region (e.g. the region except a space directly above single electrode 111, or the region above conductive layer 141) having relatively weak electric field intensity by the gradient generated by the AC field. For example, the polarized suspended substances in the mixture are driven away from the space right above single electrode 111 and therefore separated from the liquid portion of the mixture. In this situation, no or very few suspended substances in the mixture will be located right above single electrode 111.

In one embodiment, single electrode 111 is the only electrode on the chip 10.

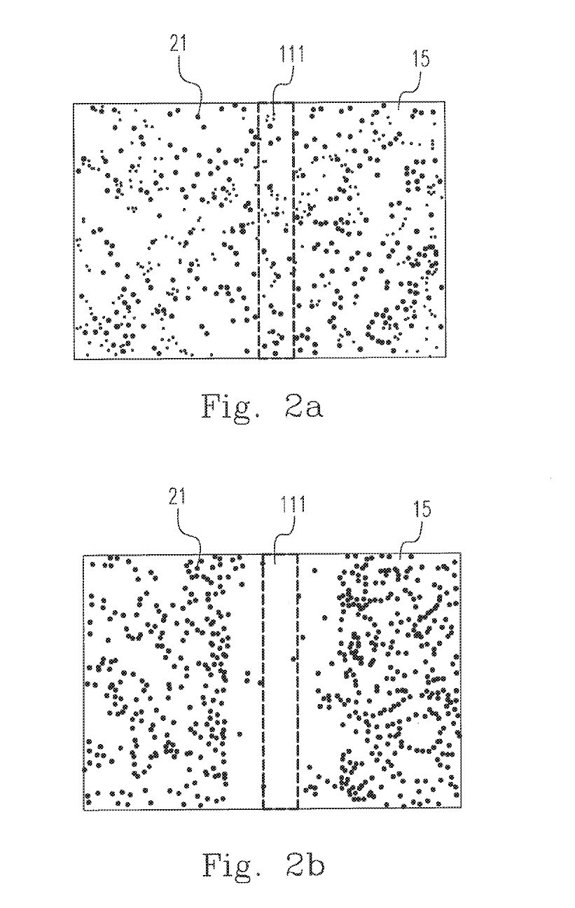

Please refer to FIGS. 2a and 2b which show an embodiment demonstrating the separation of the suspended substance and the liquid portion of a mixture by chip 10. In FIG. 2a, a mixture made of a phosphate buffer (0.1 M) and including latex beads 21 (2×10⁸ beads/mL and 6 μm in diameter) is distributed in space 15. There is no voltage applied to the mixture at this time and the latex beads 21 are uniformly distributed in the mixture in space 15 and above single electrode 111 (e.g. the region surrounded by the dotted line). FIG. 2b shows that when AC voltage (20 Vₚ₋ₚ, 100 kHz) is applied to the mixture, latex beads 21 are obviously driven away from the space right above single electrode 111, and there are almost no latex beads above single electrode 111. Therefore single electrode 111 can be seen and observed clearly without interference from the beads.

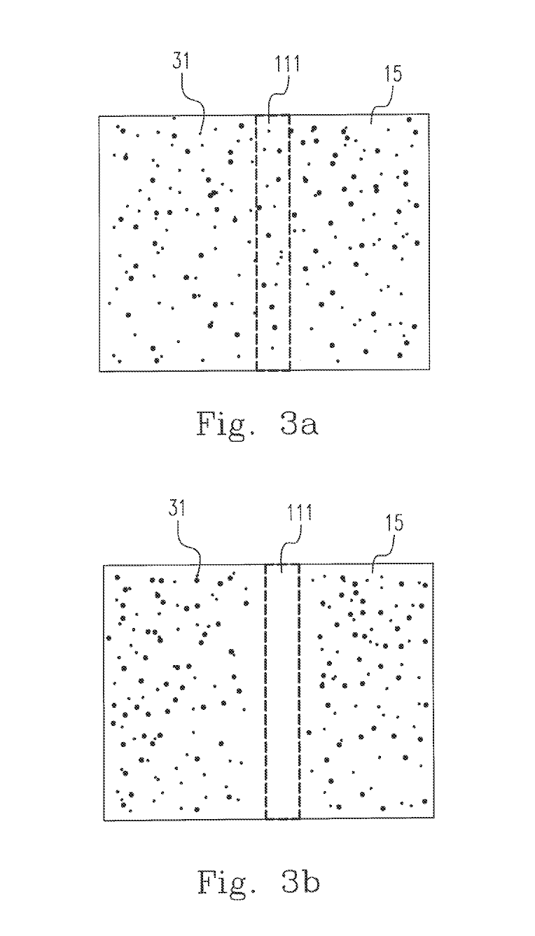

Please refer to FIGS. 3a and 3b which show an embodiment demonstrating the separation of a suspended substance and a liquid portion of a mixture by chip 10. In FIG. 3a, the mixture is made of a phosphate buffer (0.1 M), includes E. coli 31 (8×10⁸ CFU/mL) and is distributed in space 15. There is no voltage applied to the mixture at this time so the E. coli 31 are uniformly distributed in the mixture in space 15 and above single electrode 111 (i.e. the region surrounded by the dotted line). FIG. 3b shows that when AC voltage (20 Vₚ₋ₚ, 100 kHz) is applied to the mixture, E. coli 31 is driven away from the space directly right above single electrode 111 and there is almost no E. coli above single electrode 111.

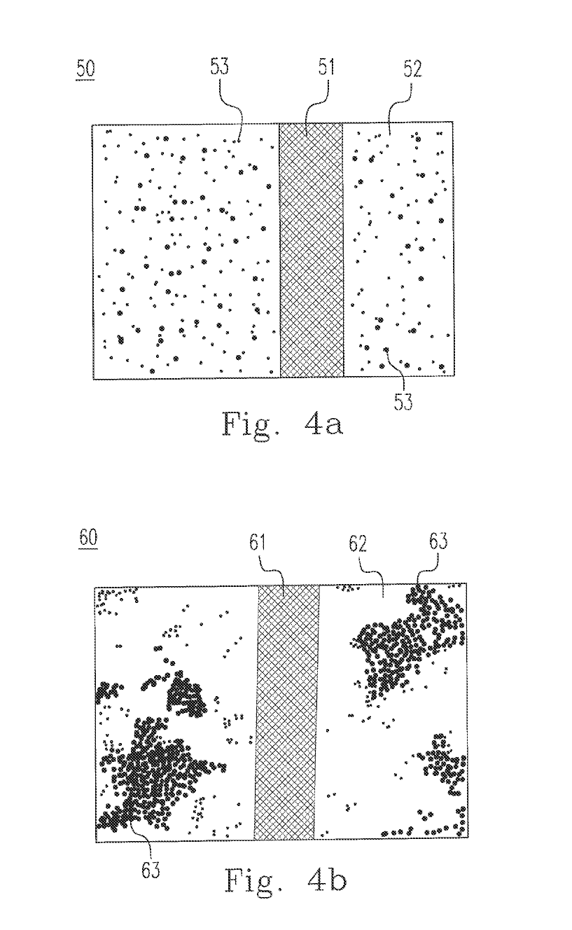

Please refer to FIGS. 4a and 4b which show embodiments demonstrating the separation and concentration of blood cells in the plasma. Specifically, blood typing is determined by the antigen on the surface of red blood cells. In the embodiments shown in FIGS. 4a and 4b, both of the chips use a gold electrode. In FIG. 4a, chip 50 includes electrode 51 and conductive layer 52, and anti-B antibody covers electrode 51 and/or conductive layer 52. When 2 μL of type A blood is dropped on single electrode 51 and conductive layer 52, AC voltage (10 Vₚ₋ₚ, 100 kHz) is then applied to the blood. Because the type A blood will not bind to the anti-B antibody covering electrode 51 and/or conductive layer 52, red blood cells 53 in the type A blood are not excluded from the region directly above electrode 51 because no agglutination is generated and observed as shown in FIG. 4a.

In FIG. 4b, chip 60 includes electrode 61 and conductive layer 62, and anti-A antibody covers electrode 51 and/or conductive layer 52. When 2 μL of type A blood (with a hematocrit of about 5%) is dropped on single electrode 61 and conductive layer 62, red blood cells 63 in the type A blood will bind with the anti-A antibody covering electrode 61 and/or conductive layer 62 so as to generate the agglutination of red blood cells 63. Because the agglutinated red blood cells 63 have larger volume than normal red blood cells, the dielectrophoretic force on the agglutinated red blood cells will be enhanced several times. Accordingly, when AC voltage (10 Vₚ₋ₚ, 100 kHz) is applied to the type A blood, the agglutinated red blood cells 63 are excluded from the region above electrode 51 and concentrated near electrode 51 to further promote the agglutination of red blood cells. Therefore, it is shown that the present chip can separate and concentrate agglutinated blood cells so as to identify the blood type and specific antigen/antibody in the blood based on the results of the blood cell removal from above the single electrode.

In some embodiments, type A blood (whole or diluted) is mixed with anti-A antibody, and then dropped on chip 60. The AC voltage is applied to the mixed type A blood to separate and concentrate the red blood cells therein, and the same result as shown in FIG. 4b will be observed.

In some embodiments, after an appropriate voltage is applied to the mixture, at least most of the suspended substances in the mixture can be excluded from above the electrode. Therefore, the liquid portion of the mixture can directly contact the electrode without the interference of the suspended substances. Moreover, if a measurement for a specific property of dissolved substance in the mixture (e.g. quantitative or qualitative analysis) is executed, the result will be more accurate then when measured in a situation where the suspended substances are not specifically separated. For example, a first indicator (called an initial indicator) which can react (e.g. specifically bind) with a specific dissolved substance in the mixture is provided. Regarding the initial indicator, it has a characteristic that, after being bound with a specific dissolved substance, it will become a bound indicator (called a changed indicator) which has a color different from that of the initial indicator. The initial indicator is modified by the electrode. Then, the mixture is dropped and distributed on the electrode, and the specific dissolved substance will react with the first indicator and cause the first indicator to generate the color change. This change can therefore serve as a reference indication or parameter for the quantitative or qualitative analysis of the specific dissolved substance.

In one embodiment, the color has a shade degree, and the concentration of the specific substance in the mixture can be obtained from the shade degree of the color. The shade degree of the color is positively related to the concentration of the specific substance. The higher the shade degree of the color is, the higher the concentration of the specific substance is.

The first indicator can be, but is not limited to, a fluorescent substance, luminescent substance, or a substance or enzyme which shows a specific color via other coloring mechanisms. The specific dissolved substance can be, but is not limited to, a protein, nucleotide, biological metabolite, metal, or environment hormone.

In some embodiments, the quantitative or qualitative analysis is performed via the binding of antigen and antibody. Specifically, a sample mixture including a suspended substance and a specific substance is dropped on the electrode on which the antibody that is able to specifically bind with the specific substance is modified. Then, an appropriate voltage is applied to the mixture and a second antibody, which can also specifically bind with the specific substance and emit fluorescent or luminescent, is added to the mixture. After the second antibody specifically binds with the specific substance and the suspended substance is driven away from above the electrode, the fluorescent or luminescent emission from the second antibody can serve as a reference indication or parameter for the quantitative or qualitative analysis of the specific substance. Moreover, because the suspended substance is driven away above the electrode, any interference from the suspended substance is reduced and so the quantitative or qualitative analysis of the specific substance is performed more quickly and the result is more accurate.

In some embodiments, the mechanism of Förster resonance energy transfer (FRET) is applicable to the binding of the indicator modified on the electrode and the specific substance in the mixture.

In some embodiments, a second indicator can be modified on the conductive layer (or further on the electrode) of the chip, where the second indicator can react (e.g. specifically bind) with the suspended substance in the mixture and therefore generate a change. When the mixture is dropped and distributed on the electrode and the conductive layer, the suspended substance will react with the second indicator and cause it to generate the change. After an appropriate voltage is applied to the mixture, the suspended substance is driven away from above the electrode and concentrated above the conductive layer. Therefore, more suspended substances are distributed above the conductive layer and bind with the second indicator so as to further enhance the change. The enhanced change can serve as a more effective reference indication or parameter for the quantitative or qualitative analysis of the suspended substance.

In some embodiments, the mixture includes a first and a second suspended substances in which the respective dielectric properties are different from each other under the same applied voltage. Thus, if appropriate voltage is applied to the mixture to generate two different dielectrophoretic forces on the first and the second suspended substances, the first and the second suspended substances will be respectively distributed on and driven away from above the electrode. If the appropriate indicator is modified on the electrode and/or the conductive layer, the first and the second suspended substances will be driven by the dielectrophoretic forces to distribute on the electrode and the conductive layer respectively and specifically bind with the indicator(s). Then, based on the change caused by the binding of the first and/or the second suspended substances and the indicator(s), an accurate quantitative and/or qualitative analysis for the first and/or the second suspended substances can be performed.

### EMBODIMENTS

Embodiment 1: a method for measuring a concentration of a specific substance in a mixture including a plurality of particles, comprising steps of providing a single electrode having an initial indicator thereon; causing the mixture to be distributed on the single electrode so that the specific substance reacts with the indicator to cause the indicator to become a changed indicator; providing a dielectrophoretic force acting on the plurality of particles by the single electrode to drive at least a part of the plurality of particles away from a space directly above the single electrode; and obtaining the concentration of the specific substance via the changed indicator.

Embodiment 2 is a method as described in Embodiment 1, where the plurality of particles are microorganisms.

Embodiment 3 is a method as described in Embodiment 1 or 2, where the specific substance is one selected from the group consisting of a protein, a nucleotide, a metabolite of the microorganisms and the combination thereof.

Embodiment 4 is a method as described in Embodiments 1 to 3, where the initial indicator is specifically bound with the specific substance so as to become the changed indicator, and the changed indicator has a color different from that of the initial indicator.

Embodiment 5 is a method as described in Embodiment 4, where the color has a shade degree, and the concentration of the specific substance is obtained from the shade degree of the color.

Embodiment 6 is a method as described in Embodiments 1 to 5 and further comprises steps of applying an AC current to the single electrode to generate the dielectrophoretic force; obtaining an electric property of the mixture using the single electrode having the changed indicator thereon; and obtaining the concentration of the specific substance through the electric property.

Embodiment 7 is a method as described in Embodiment 6, where the electric property is an impedance.

Embodiment 8 is a method as described in Embodiments 1 to 7, where the mixture is blood, and the plurality of particles are blood cells.

Embodiment 9 is a method as described in Embodiments 1 to 8, where the mixture is wastewater, and the specific substance is a heavy metal.

Embodiment 10 is a method as described in Embodiments 1 to 9, where the specific substance is soluble in the mixture.

Embodiment 11: a measuring apparatus, comprising a carrying portion carrying a liquid mixture, where the liquid mixture includes a non-dissoluble substance and a dissolved substance, the carrying portion includes an electrode having a surface directly contacting the liquid mixture and having an indicator thereon, and the indicator reacts with the dissolved substance; and a conductive layer directly contacting the liquid mixture, where the electrode and the conductive layer generate an AC field in the liquid mixture so as to cause the non-dissoluble substance to be distributed on the conductive layer, the dissolved substance reacts with the indicator to cause the indicator to become a changed indicator, and the changed indicator reflects a property of the dissolved substance.

Embodiment 12 is a method as described in Embodiment 11, where the apparatus has only one electrode being the electrode, the electrode is electrically connected to a computing device, the non-dissolved substance is non-dissolved and suspended in the liquid mixture, the dissolved substance is dissolved in the liquid mixture and has a concentration in the liquid mixture, the property is the concentration of the dissolved substance in the liquid mixture, the changed indicator has an amount, and the computing device calculates the concentration of the dissolved substance based on the amount of the changed indicator.

Embodiment 13: a measuring method, comprising steps of providing an electrode and a conductive layer, wherein at least one of the electrode and the conductive layer has a first indicator distributed thereon; providing a mixture including a suspended substance; distributing the mixture on the electrode and the conductive layer, wherein the suspended substance reacts with the first indicator to cause the first indicator to become a first changed indicator; generating an AC field in the mixture via the electrode and the conductive layer to drive the suspended substance away from a space directly above the electrode; and obtaining a property of the suspended substance based on the first changed indicator.

Embodiment 14 is a method as described in Embodiment 13, where the second indicator is specifically bound with the dissolved substance so as to become the second changed indicator, the method further comprising steps of obtaining an electric property of the mixture using the electrode having the second changed indicator thereon; and obtaining the concentration of the dissolved substance from the electric property.

Embodiment 15 is a method as described in Embodiment 13 or 14, where the mixture further includes a dissolved substance, the electrode includes a second indicator distributed thereon, the dissolved substance reacts with the second indicator to cause the second indicator to become a second changed indicator, the dissolved substance has a concentration in the mixture, the second changed indicator has an amount, and the method further comprises a step of obtaining the concentration of the dissolved substance in the mixture based on the amount of the second changed indicator.

Embodiment 16 is a method as described in Embodiments 13 to 15, where the first indicator is specifically bound with the suspended substance to become the first changed indicator, and the suspended substance specifically bound with the first indicator is driven away from the space directly above the electrode.

Embodiment 17 is a method as described in Embodiments 13 to 16, where the first indicator is an antibody specifically bound with the suspended substance, and the suspended substance is a red blood cell.

Embodiment 18 is a method as described in Embodiments 13 to 17, where the AC field generates a dielectrophoretic force acting on the suspended substance to cause the suspended substance to be driven away from the space directly above the electrode.

Embodiment 19 is a method as described in Embodiment 13 to 18, where the first indicator is specifically bound with the suspended substance to become the first changed indicator.

While the disclosure has been described in terms of what is presently considered to be the most practical and preferred embodiments, it is to be understood that the disclosure is not limited to the disclosed embodiments. Therefore, it is intended to cover various modifications and similar arrangements included within the spirit and scope of the appended claims, which are to be accorded with the broadest interpretation so as to encompass all such modifications and similar structures.

What is claimed is:

1. A method for measuring a concentration of a specific substance in a mixture including a plurality of particles, comprising steps of:
providing a single electrode having an initial indicator thereon;
causing the mixture to be distributed on the single electrode so that the specific substance reacts with the indicator to cause the indicator to become a changed indicator;
providing a dielectrophoretic force acting on the plurality of particles by the single electrode to drive at least a part of the plurality of particles away from a space directly above the single electrode; and
obtaining the concentration of the specific substance via the changed indicator.

2. The method as claimed in claim 1, wherein the plurality of particles are microorganisms.

3. The method as claimed in claim 2, wherein the specific substance is one selected from the group consisting of a protein, a nucleotide, a metabolite of the microorganisms and the combination thereof.

4. The method as claimed in claim 1, wherein the indicator is specifically bound with the specific substance so as to become the changed indicator, and the changed indicator has a color different from that of the initial indicator.

5. The method as claimed in claim 1, wherein the color has a shade degree, and the concentration of the specific substance is obtained from the shade degree of the color.

6. The method as claimed in claim 1, further comprising steps of:
applying an AC current to the single electrode to generate the dielectrophoretic force;
obtaining an electric property of the mixture using the single electrode having the changed indicator thereon; and
obtaining the concentration of the specific substance through the electric property.

7. The method as claimed in claim 6, wherein the electric property is an impedance.

8. The method as claimed in claim 1, wherein the mixture is a blood, and the plurality of particles are blood cells.

9. The method as claimed in claim 1, wherein the mixture is a wastewater, and the specific substance is a heavy metal.

10. The method as claimed in claim 1, wherein the specific substance is soluble in the mixture.

11. A measuring apparatus, comprising:
a carrying portion carrying a liquid mixture, wherein the liquid mixture includes a non-dissoluble substance and a dissolved substance, and the carrying portion includes:
an electrode having a surface directly contacting the mixture and having an indicator thereon, wherein the indicator reacts with the dissolved substance; and
a conductive layer directly contacting the liquid mixture, wherein the electrode and the conductive layer generate an AC field in the liquid mixture so as to cause the non-dissoluble substance to be distributed on the conductive layer, the dissolved substance reacts with the indicator to cause the indicator to become a changed indicator, and the changed indicator reflects a property of the dissolved substance.

12. The apparatus as claimed in claim 11, wherein the apparatus has only one electrode being the electrode, the electrode is electrically connected to a computing device, the non-dissolved substance is non-dissolved and suspended in the liquid mixture, the dissolved substance is dissolved in the liquid mixture and has a concentration in the liquid mixture, the property is the concentration of the dissolved substance in the liquid mixture, the changed indicator has an amount, and the computing device calculates the concentration of the dissolved substance based on the amount of the changed indicator.

13. A measuring method, comprising steps of:
providing an electrode and a conductive layer, wherein at least one of the electrode and the conductive layer has a first indicator distributed thereon;
providing a mixture including a suspended substance;
distributing the mixture on the electrode and the conductive layer, wherein the suspended substance reacts with the first indicator to cause the first indicator to become a first changed indicator;
generating an AC field in the mixture via the electrode and the conductive layer to drive the suspended substance away from a space directly above the electrode; and
obtaining a property of the suspended substance based on the first changed indicator.

14. The method as claimed in claim 14, wherein the second indicator is specifically bound with the dissolved substance so as to become the second changed indicator, the method further comprising steps of:
obtaining an electric property of the mixture using the electrode having the second changed indicator thereon; and
obtaining the concentration of the dissolved substance from the electric property.

15. The measuring method as claimed in claim 13, wherein the mixture further includes a dissolved substance, the electrode includes a second indicator distributed thereon, the dissolved substance reacts with the second indicator to cause the second indicator to become a second changed indicator, the dissolved substance has a concentration in the mixture, the second changed indicator has an amount, and the method further comprises a step of:
obtaining the concentration of the dissolved substance in the mixture based on the amount of the second changed indicator.

16. The measuring method as claimed in claim 13, wherein the first indicator is specifically bound with the suspended substance to become the first changed indicator, and the suspended substance specifically bound with the first indicator is driven out of the space right above the electrode.

17. The measuring method as claimed in claim 13, wherein the first indicator is an antibody specifically bound with the suspended substance, and the suspended substance is red blood cells.

18. The measuring method as claimed in claim 13, wherein the AC field generates a dielectrophoretic force acting on the suspended substance to cause the suspended substance to be driven out of the space right above the electrode.

19. The measuring method as claimed in claim 13, wherein the first indicator is specifically bound with the suspended substance to become the first changed indicator.
